(19) United States
(12) Patent Application Publication
(10) Pub. No.: US 2024/0269141 A1
(43) Pub. Date: Aug. 15, 2024

(54) NADES FORMULATIONS COMPRISING PHARMACEUTICALS

(71) Applicant: Clemson University, Clemson, SC (US)

(72) Inventors: Carlos D. Garcia, Clemson, SC (US); Lucas Ayres, Clemson, SC (US)

(21) Appl. No.: 18/431,215

(22) Filed: Feb. 2, 2024

Related U.S. Application Data
(60) Provisional application No. 63/443,481, filed on Feb. 6, 2023.
(60) Provisional application No. 63/447,772, filed on Feb. 23, 2023.
(60) Provisional application No. 63/450,275, filed on Mar. 6, 2023.
(60) Provisional application No. 63/453,939, filed on Mar. 22, 2023.

(51) Int. Cl. A61K 31/519; A61K 31/122; A61K 31/138; A61K 47/02; A61K 47/08; A61K 47/10; A61K 47/12; A61K 47/18; A61K 47/20; A61K 47/26

(52) U.S. Cl. CPC A61K 31/519; A61K 31/122; A61K 31/138; A61K 47/02; A61K 47/08; A61K 47/10; A61K 47/12; A61K 47/18; A61K 47/183; A61K 47/186; A61K 47/20; A61K 47/26

(57) ABSTRACT

A eutectic mixture comprising a pharmaceutically active ingredient and a least one compound selected from an amine or alkyl amine and an alcohol or a carboxylic acid, wherein said eutectic mixture is a liquid at a given temperature where at least one of its components would, otherwise, be a solid unfit to be applied as a solvent.

### CROSS-REFERENCE TO RELATED APPLICATIONS

The present application claims priority to pending U.S. Provisional Application Nos. 63/443,481 filed Feb. 6, 2023; 63/447,772 filed Feb. 23, 2023; 63/450,275 filed Mar. 6, 2023 and 63/453,939 filed Mar. 22, 2023 each of which is incorporated herein by reference.

### FIELD OF THE INVENTION

The present invention is related to formulations of novel natural deep eutectic solvents (NADES) comprising pharmaceutically active materials; particularly ibrutinib, sildenafil derivatives, ubiquinone derivatives or tamoxifen derivatives; as a component of the NADES. The application is also related to pharmaceutical compositions comprising the eutectic mixtures.

### BACKGROUND

Eutectic mixtures such as ionic liquids (IL), deep eutectic solvents (DES) and natural deep eutectic solvents (NADES) have been extensively studied as green alternative liquid media to traditional organic solvents. For the purposes of the present invention the primary focus will be on NADES without limit thereto.

DES and NADES are formed from specific ratios of multiple, typically two or three, components leading to a system that is a liquid at a given temperature where at least one of its components would, otherwise, be a solid unfit to be applied as a solvent. The formation of eutectic solvents typically involves the formation of intrinsic interactions between the components, for example by moderate heating and stirring, rarely obtained by simply mixing the components. Of particular interest, without limit thereto, are those mixtures comprising components which, when formed into a stable eutectic mixture, feature a melting point sufficiently low to be a liquid at ambient temperature, such as 20-30° C.

Eutectic mixtures have proven to be very beneficial for use in extracting various materials, for solubilizing various materials, and as excipients for several active pharmaceutical ingredients. The present invention is related to the extension of eutectics, and particularly NADES, that include pharmaceutically active ingredients (API) as a component of the NADES, wherein the melting point of the NADES incorporating the pharmaceutically active ingredient as one of its components is significantly low to be a liquid at ambient temperature and that typically feature better pharmacokinetic and/or pharmacodynamic profiles than the original pharmaceutically active ingredient.

### SUMMARY OF THE INVENTION

The present invention is related to the formation of a eutectic mixture comprising a pharmaceutically active ingredient as a component of the eutectic mixture wherein the eutectic mixture has a melting point lower than the pharmaceutically active ingredient, resulting in a liquid at ambient temperature which is defined herein as 20-30° C.

A particular feature of the instant invention is the formation of a eutectic mixture comprising pharmaceuticals; particularly ibrutinib, sildenafil derivatives, ubiquinone derivatives or tamoxifen derivatives; as a component of the eutectic mixture.

A particular advantage of the instant invention is the ability to provide a formulation comprising the eutectic mixture with other components which are not a component of the eutectic mixture.

A particular advantage of the present invention is the ability to form a eutectic mixture of pharmaceuticals; particularly ibrutinib, sildenafil derivatives, ubiquinone derivatives or tamoxifen derivatives; wherein the pharmaceutical forming the eutectic mixture is more readily delivered to mucosal membranes for transport through the mucosal membranes than as ibrutinib alone or ibrutinib simply dissolved in other substances.

These, and other advantages, as will be realized, are provided in a eutectic mixture comprising a pharmaceutically active material and a least one compound selected from an amine or alkyl amine and an alcohol or a carboxylic acid. The eutectic mixture has a melting point low enough to remain liquid at ambient temperature.

Yet another embodiment is provided in pharmaceutical composition comprising a eutectic mixture comprising a pharmaceutically active material and a least one compound selected from an amine or alkyl amine and an alcohol or a carboxylic acid. The eutectic mixture has a melting point low enough to remain liquid at ambient temperature.

### DESCRIPTION

The present invention is related to a eutectic mixture comprising pharmaceuticals; particularly ibrutinib, sildenafil derivatives, ubiquinone derivatives or tamoxifen derivatives; as a component of the eutectic mixture. The eutectic mixture has a melting point which is low enough to remain liquid at ambient temperature. The eutectic mixture may be incorporated into a formulation comprising other components, such as solvents, which may or may not disrupt the intermolecular interactions of the eutectic mixture and therefore may or may not disrupt the eutectic mixture.

The inventive eutectic mixture comprises pharmaceuticals; particularly ibrutinib, a sildenafil derivative, a ubiquinone derivative or a tamoxifen derivative; and at least one of an alcohol or carboxylic acid and an amine or alkyl amine, preferably a compound containing both functionalities, such as alkynolamine. Formation of the eutectic mixture comprises mixing of the components, at a specific stoichiometric ratio, followed by introduction of energy. The amount of energy must be sufficient to disrupt the intermolecular interactions in the reactants, sometimes in solid forms, leading to new intermolecular interactions between the pharmaceuticals; particularly ibrutinib, a sildenafil derivative, a ubiquinone derivative or a tamoxifen derivative; alcohol or carboxylic acid and amine or alkyl amine forming the eutectic mixture. Thermal processes are typically sufficient such as heating in a sealed vial, with agitation, at about 80° C. or more for an amount of time sufficient to promote the formation of the eutectic mixture. Other techniques can be employed to form the eutectic mixture such as ultrasonic mixing, microwave heating, solvent evaporation and the like.

It is particularly preferred that the eutectic mixture is stable for at least one week wherein stable is defined as a eutectic mixture which does not separate into the components when left at ambient temperature for at least one week.

Ibrutinib is represented by General Formula A:

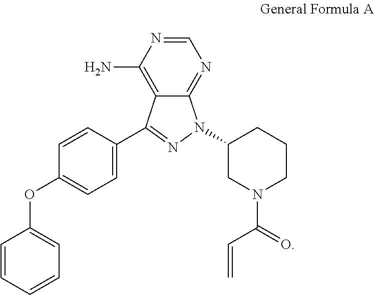

Particularly preferred embodiments comprising ibrutinib are selected from the group consisting of: 0.9-1.1 part choline chloride, 4.5-5.5 parts laurinol, 2.7-3.3 parts ibrutinib and 4.5-5.5 parts urea; 1.8-2.2 parts choline chloride, 4.5-5.5 parts ethyl-(2-hydroxyethyl)-dimethylazanium chloride, 4.5-5.5 parts dichlorozinc and 0.9-1.1 part ibrutinib; 0.9-1.1 part choline chloride, 0.9-1.1 part undec-10-enoic acid, 1.8-2.2 parts choline fluoride and 2.7-3.3 parts ibrutinib; 0.9-1.1 part choline chloride, 1.8-2.2 parts 1,3,4,5,6-pentahydroxyhexan-2-one, 0.9-1.1 part butane-1,4-diol and 4.5-5.5 parts ibrutinib; 0.9-1.1 part choline chloride, 2.7-3.3 parts octanoic acid, 1.8-2.2 parts ibrutinib, and 0.9-1.1 part benzyl(trimethyl)azanium chloride; 0.9-1.1 part camphor, 2.7-3.3 parts laurinol, 1.8-2.2 parts boric acid and 4.5-5.5 parts ibrutinib; 0.9-1.1 part choline chloride, 0.9-1.1 part nonanoic acid, 4.5-5.5 parts sodium acetate and 4.5-5.5 parts ibrutinib; 0.9-1.1 part borneol, 4.5-5.5 parts ethyl-(2-hydroxyethyl)-dimethylazanium chloride, 4.5-5.5 parts palmitic acid, 0.9-1.1 part lactose and 3.6-4.4 parts ibrutinib; 0.9-1.1 part choline chloride, 1.8-2.2 parts hexanoic acid, 2.7-3.3 parts ibrutinib, 2.7-3.3 parts boric acid and 0.9-1.1 part ethyl(2-hydroxyethyl)dimethylammonium chloride; and 0.9-1.1 part choline chloride, 2.7-3.3 parts ibrutinib, 1.8-2.2 parts 2-(diethylamino)ethanol hydrochloride and 2.7-3.3 parts hexanoic acid.

A sildenafil derivative is represented by General Formula B:

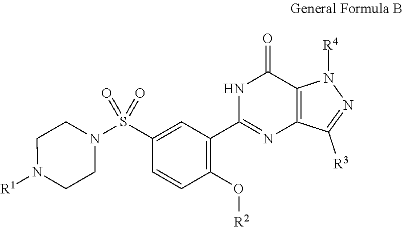

wherein:
R¹-R⁴ are independently selected from H or an alkyl of 1-10 carbons; alkenyl of up to 10 carbons.
R¹ is preferably H or an alkyl of 1-3 carbons and most preferably —CH₃.
R² is preferably H or an alkyl of 1-3 carbons and most preferably —CH₂CH₃.
R³ is preferably H or an alkyl of 1-4 carbons and most preferably —CH₂CH₂CH₃.
R⁴ is preferably H or an alkyl of 1-3 carbons and most preferably —CH₃.

In a particularly preferred embodiment R¹ is —CH₃, R² is —CH₂CH₃, R³ is —CH₂CH₂CH₃ and R⁴ is —CH₃ which is referred to in the art as Sidenafil sold under the brand name Viagra®.

Particularly preferred embodiments comprising sildenafil derivatives are selected from the group consisting of: 2.7-3.3 parts choline chloride, 1.8-2.2 parts alpha-maltose 2.7-3.3 parts sildenafil and 2.7-3.3 parts (diethylamino)ethanol hydrochloride; 4.5-5.5 parts choline chloride, 3.6-4.4 parts laurinol, 2.7-3.3 parts benzyltriethylammonium chloride, 1.8-2.2 parts metilotic acid and 3.6-4.4 parts sildenafil; 4.5-5.5 parts choline fluoride, 1.8-2.2 parts glucose, 0.9-1.1 part laurinol, 1.8-2.2 parts tetradecanoic acid and 4.5-5.5 parts sildenafil; 0.9-1.1 part triethanolamine, 0.9-1.1 part ammonium rhodanide, 0.9-1.1 part sildenafil and 0.9-1.1 part 1,3-propanediol; 1.8-2.2 parts choline chloride, 1.8-2.2 parts galactose, 1 part DL-threonine, 1.8-2.2 parts menthol and 0.9-1.1 part sildenafil; 0.9-1.1 part ethylazanium chloride, 1.8-2.2 parts vanillin, 0.9-1.1 part alpha-D-glucopyranoside, 4.5-5.5 parts alpha-maltose and 0.9-1.1 part sildenafil; 3.6-4.4 parts choline chloride, 3.6-4.4 parts laurinol and 2.7-3.3 parts sildenafil; 0.9-1.1 part borneol, 3.6-4.4 parts D-ribose, 0.9-1.1 part laurinol and 4.5-5.5 parts sildenafil; 0.9-1.1 part choline chloride, 0.9-1.1 part glutamic acid and 4.5-5.5 parts sildenafil; and 0.9-1.1 part choline chloride, 1.8-2.2 parts sildenafil, 2.7-3.3 parts borneol, 2.7-3.3 parts hexafluoroisopropanol and 2.7-3.3 parts trimesic acid. Ubiquinone is represented by General Formula C:

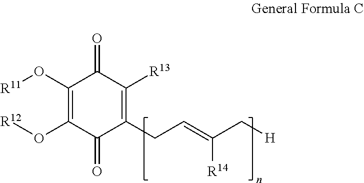

wherein R¹¹, R¹², R¹³ and R¹⁴ are independently selected from H, and an alkyl of 1-10 carbons, more preferably an alkyl of 1-5 carbons and most preferably —CH₃; and n is an integer of 1-15, more preferably 5-15, even more preferably 9-11 and most preferably 10.

Tamoxifen derivatives are represented by General Formula D:

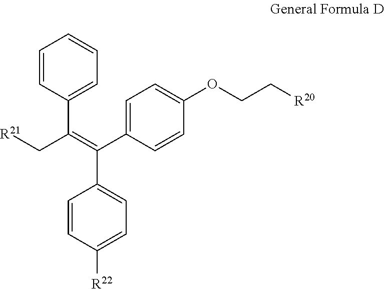

wherein:
R²⁰ is NR²³R²⁴ or OR²⁵;
R²¹ is CH₂R²⁶;
R²² is —H or —OH;
R²³ and R²⁴ are independently selected from H and an alkyl of 1-5 carbons and most preferably —CH₃;
R²⁵ is —H or an alkyl of 1-5 carbons and preferably H; and
R²⁶ is —H, —CI or —OH.

In the General Formula D when R is —N(CH₃)₂, R²¹ is —CH₃ and R²² is —H, the compound is tamoxifen. In the General Formula D when R²⁰ is NHCH₃, R²¹ is —CH₃ and R²² is —OH the compound is endoxifen. In the General Formula D when R²⁰ is NHCH₃, R²¹ is —CH₃ and R²² is —H the compound is N-desmethylamoxifen. In the General Formula D when R²⁰ is NH₂, R²¹ is —CH₃ and R²² is —H the compound is N,N-didesmethylamoxifen. In the General Formula D when R²⁰ is —OH, R²¹ is —CH₂Cl and R²² is —H the compound is ospemifene. In the General Formula D when R²⁰ is —N(CH₃)₂, R²¹ is —CH₃ and R²² is —OH the compound is afimoxifene.

Particularly preferred alcohols or carboxylic acids include mono-alcohols such as methanol, propane-1-ol, hexafluoroisopropanolmenthol, tetradecane-1-ol, 1-tetradecanol, borneol or vanillin; polyols such as ethylene glycol, glycerol, ethane-1,2-diol, butane-1,2-diol, butane-1,3-diol, 1,4 butanediol, butane-1,2,3,4-tetrol, 1,3-propanediol, 1,6-hexanediol, pentinol, hexitol or ribose particularly D-ribose; sugars such as glucose, galactose, lactose, maltose particularly alpha maltose and alpha-D-glucopyranoside; carboxylic acids such as tetradecanoic acid, 2-aminopentanedioic acid, lauric acid, nonanoic acid, formic acid, dodecanoic acid, adipic acid, stearic acid, phthalic acid, undec-10-enoic acid, undecanoic acid, 1,3,4,5,6-pentahydroxyhexan-2-one, glutamic acid, octanoic acid, palmitic acid, hexanoic acid, trimesic acid and combinations thereof such as sorbose or laurinol.

Ketones such as camphor or nonanol are also suitable for use in a eutectic.

A preferred amine is urea. The alkyl amine is preferably an ethyl amine, more preferably an alkynol amine most preferably comprising an ethanol amine (HOCH₂CH₂N—) group. A particularly preferred alkyl amine is 1-(diethylamino)ethanol hydrochloride or threonine and particularly DL-threonine.

Particularly preferred alkyl amines include azanium halides and particularly alkyl azanium halides such as ethylazanium chloride or tetraethylazanium chloride, choline halide and particularly choline chloride or choline fluoride, ethyl-(2-hydroxyethyl)-dimethylazanium chloride, diethyl-(2-hydroxyethyl)-azanium chloride, benzyl(triethyl)azanium chloride, benzyl(trimethyl)azanium chloride and tetrabutylazanium chloride; 2-[bis(2-hydroxyethyl)amino]ethanol, 2-(2-hydroxyethylamino)ethanol, 2-aminoethanol, tetraethylamine chloride, 1,3 dimethyl urea, 1,1-dimethyl urea, trimethyl glycine (betaine), carnitine, ethyl(2-hydroxyethyl)dimethylammonium chloride, diethanolamine and ethanol amine.

Additional components of the eutectic mixture include bases such as ammonium thiocyanate, dichlorozinc, boric acid, sodium acetate and eucalyptol.

It is particularly preferred to utilize the eutectic mixture in a pharmaceutical formulation comprising 1 to 99 wt % carriers and additional components. Particularly preferred carriers include solvents, particularly oils and most particularly natural oils. Additional components comprise active ingredients such as cough suppressants, aromatics, analgesics and the like for multi-purpose pharmaceuticals. Eucalyptol and eucalyptus oil are particularly suitable components of the pharmaceutical formulation.

The eutectic is in a stoichiometric relationship. Therefore, the molar ratio of pharmaceutical to each other component of a binary eutectic are typically at least 1:10 to no more than 10:1 and more preferably at least 1:5 to no more than 5:1. Mixtures containing more than two components (such as ternary quaternary, etc.) are also possible.

Exemplary embodiments comprising ibrutinib are listed in Table 1.

TABLE 1

| # | Components |
|---|---|
| 1 | 1 part choline chloride, 5 parts laurinol 3 parts ibrutinib and 5 parts |
| | urea |
| 2 | 2 parts choline chloride, 5 parts ethyl-(2-hydroxyethyl)- |
| | dimethylazanium chloride, 5 parts dichlorozinc and 1 part ibrutinib |
| 3 | 1 part choline chloride, 1 part undec-10-enoic acid, 2 parts choline |
| | fluoride and 3 parts ibrutinib |
| 4 | 1 part choline chloride, 2 parts 1,3,4,5,6-pentahydroxyhexan-2-one, 1 |
| | part butane-1,4-diol and 5 parts ibrutinib |
| 5 | 1 part choline chloride, 3 parts octanoic acid, 2 parts ibrutinib, and 1 |
| | part benzyl(trimethyl)azanium chloride |
| 6 | 1 part camphor, 3 parts laurinol, 2 parts boric acid and 5 parts |
| | ibrutinib |
| 7 | 1 part choline chloride, 1 part nonanoic acid, 5 parts sodium acetate |
| | and 5 parts ibrutinib |
| 8 | 1 part borneol, 5 parts ethyl-(2-hydroxyethyl)-dimethylazanium |
| | chloride, 5 parts palmitic acid, 1 part lactose and 4 parts ibrutinib |
| 9 | 1 part choline chloride, 2 parts hexanoic acid, 3 parts ibrutinib, 3 |
| | parts boric acid and 1 part ethyl(2-hydroxyethyl)dimethylammonium |
| | chloride |
| 10 | 1 part choline chloride, 3 parts ibrutinib, 2 parts 2- |
| | (diethylamino)ethanol hydrochloride and 3 parts hexanoic acid |

Exemplary embodiments comprising a sildenafil derivative are listed in Table 2.

TABLE 2

| # | Components |
|---|---|
| 1 | 3 parts choline chloride, 2 parts alpha-maltose 3 parts sildenafil and 3 |
| | parts (diethylamino)ethanol hydrochloride |
| 2 | 5 parts choline chloride, 4 parts laurinol, 3 parts |
| | benzyltriethylammonium chloride, 2 parts metilotic acid and 4 parts |
| | sildenafil |
| 3 | 5 parts choline fluoride, 2 parts glucose, 1 part laurinol, 2 parts |
| | tetradecanoic acid and 5 parts sildenafil |
| 4 | 1 part triethanolamine, 1 part ammonium rhodanide (thiocyanate), 1 |
| | part sildenafil and 1 part 1,3, propanediol |
| 5 | 2 parts choline chloride, 2 parts galactose, 1 part DL-threonine, 2 |
| | parts menthol and 1 part sildenafil |
| 6 | 1 part ethylazanium chloride, 2 parts vanillin, 1 part alpha-D- |
| | glucopyranoside, 5 parts alpha-maltose and 1 part sildenafil |
| 7 | 4 parts choline chloride, 4 parts laurinol and 3 parts sildenafil |
| 8 | 1 part borneol, 4 parts D-ribose, 1 part laurinol and 5 parts sildenafil |
| 9 | 1 part choline chloride, 1 part glutamic acid and 5 parts sildenafil |
| 10 | 1 part choline chloride, 2 parts sildenafil, 3 parts borneol 3 parts |
| | hexafluoroisopropanol and 3 parts trimesic acid |

Exemplary embodiments comprising a ubiquinone derivative are listed in Table 3.

TABLE 3

| # | Components |
|---|---|
| 1 | 1 part choline chloride, 2 parts ubiquinone and 4 parts glycerol |
| 2 | 1 part ethylazanium chloride, 1 part butane-1,4-diol, 2 parts 1,1- |
| | dimethylurea, 1 part laurinol and 4 parts ubiquinone |
| 3 | 1 part choline chloride, 3 parts ubiquinone and 1 part glucose |
| 4 | 1 part choline chloride, 3 parts ubiquinone and 2 parts 1,3- |
| | dimethylurea |
| 5 | 1 part choline chloride, 2 parts maltose, 2 parts 1,3-dimethylurea, 1 |
| | part ubiquinone and 4 parts camphor |
| 6 | 5 parts camphor, 1 parts ubiquinone, 1 part butane-1,2,3,4-tetrol |
| | and 1 part ethane-1,2-diol |
| 7 | 5 parts choline chloride, 1 part ubiquinone and 2 parts dodecanoic |
| | acid |
| 8 | 1 part choline chloride, 4 parts alpha-maltose, 3 parts ubiquinone, |
| | and 4 parts phthalic acid |
| 9 | 2 parts choline chloride, 1 part 2-(trimethylazaniumyl)acetate, 5 parts |
| | ethyl-(2-hydroxyethyl)-dimethylazanium chloride, 5 parts ubiquinone |
| | and 3 parts nonanal |
| 10 | 1 part camphor, 3 parts ubiquinone, 1 part ethylene glycol |

In Table 3, ubiquinone is represented by the General Formula C with R¹¹, R¹², R¹³ and R¹⁴ being —CH₃ and n being 10.

Exemplary embodiments comprising a tamoxifen derivative are listed in Table 4.

TABLE 4

| # | Components |
|---|---|
| 1 | 1 part choline chloride, 5 parts tamoxifen, 3 parts hexitol, 1 part |
| | ethylene glycol and 1 part tetraethylazanium chloride |
| 2 | 1 part choline chloride, 1 part tamoxifen, 3 parts sodium acetate, 3 |
| | parts formic acid and 1 part butane-1,3-diol |
| 3 | 1 part choline chloride, 1 part tamoxifen, 2 parts choline fluoride, 5 |
| | parts hexanoic acid and 2 parts 2-aminoethanol |
| 4 | 1 part choline chloride, 4 parts diethyl(2-hydroxyethyl) azanium |
| | chloride, 3 parts diethanolamine and 2 parts tamoxifen |
| 5 | 1 part ethylazanium chloride, 1 part 1,2-butanediol, 2 parts |
| | methanol, 3 parts laurinol and 5 parts tamoxifen |
| 6 | 2 parts choline chloride, 2 parts tamoxifen and 3 parts choline |
| | flouride |
| 7 | 3 parts choline chloride, 1 part tamoxifen and 3 parts glycerol |
| 8 | 3 parts choline chloride, 4 parts tamoxifen and 3 parts carnitine |
| 9 | 3 parts choline chloride, 1 part tamoxifen and 4 parts butane-1,3-diol |
| 10 | 1 part choline chloride, 1 part hexanoic acid and 3 parts tamoxifen |

In the Tables “part” refers to the molar ratio of the component wherein the molar ratio is ±10%. By way of example, a composition listing 1 part A, 2 parts B and 5 parts C indicates 0.9-1.1 part A, 1.8-2.2 parts B and 4.5-5.5 parts C.

The invention has been described with reference to preferred embodiments without limit thereto. One of skill in the art would realize additional embodiments which are described and set forth in the claims appended hereto.

Claimed is:

1. A eutectic mixture comprising:
a pharmaceutically active material and a least one compound selected from an amine or alkyl amine and an alcohol or a carboxylic acid.
wherein said eutectic mixture has a melting point low enough to remain liquid at ambient temperature.

2. The eutectic mixture of claim 1 comprising an amine or alkyl amine.

3. The eutectic mixture of claim 2 wherein said amine is urea.

4. The eutectic mixture of claim 2 wherein said alkyl amine is an alkynol amine.

5. The eutectic mixture of claim 4 wherein said alkyl amine is selected from the group consisting of 1-(diethylamino)ethanol hydrochloride and threonine.

6. The eutectic mixture of claim 2 wherein said alkyl amine is selected from the group consisting of azanium halides; 2-[bis(2-hydroxyethyl)amino]ethanol, 2-(2-hydroxyethylamino)ethanol, tetraethylamine chloride, 1,3 dimethyl urea, trimethyl glycine (betaine), ethyl(2-hydroxyethyl)dimethylammonium chloride and ethanol amine.

7. The eutectic mixture of claim 6 wherein said azanium halides is selected from the group consisting of ethylazanium chloride, choline halide and particularly choline chloride, ethyl-(2-hydroxyethyl)-dimethylazanium chloride, benzyl(triethyl)azanium chloride, benzyl(trimethyl)azanium chloride and tetrabutylazanium chloride; 2-[bis(2-hydroxyethyl)amino]ethanol, 2-(2-hydroxyethylamino)ethanol, tetraethylamine chloride, 1,3 dimethyl urea, trimethyl glycine (betaine) and ethanol amine.

8. The eutectic mixture of claim 1 comprising an alcohol.

9. The eutectic mixture of claim 8 wherein said alcohol is selected from a mono-alcohol and a polyol.

10. The eutectic mixture of claim 9 wherein said alcohol is selected from the group consisting of propane-1-ol, hexafluoroisopropanolmenthol, tetradecane-1-ol, 1-tetradecanol, borneol, vanillin, ethylene glycol, glycerol, butane-1,2-diol, 1,4 butanediol, 1,3-propanediol, 1,6-hexanediol, 1,3,4,5,6-pentahydroxyhexan-2-one, pentinol, ribose, glucose, galactose, lactose, maltose and alpha-D-glucopyranoside.

11. The eutectic mixture of claim 1 comprising a carboxylic acid.

12. The eutectic mixture of claim 11 wherein said carboxylic acid is selected from the group consisting of tetradecanoic acid, 2-aminopentanedioic acid, lauric acid, nonanoic acid, formic acid, dodecanoic acid, adipic acid, stearic acid, undec-10-enoic acid, glutamic acid, palmitic acid, hexanoic acid, trimesic acid, octanoic acid, sorbose and laurinol.

13. The eutectic mixture of claim 1 wherein said pharmaceutically active material is selected from the group consisting of ibrutinib, a sildenafil derivative, a ubiquinone derivative and a tamoxifen derivative.

14. The eutectic mixture of claim 13 selected from the group consisting of: 0.9-1.1 part choline chloride, 4.5-5.5 parts laurinol, 2.7-3.3 parts ibrutinib and 4.5-5.5 parts urea; 1.8-2.2 parts choline chloride, 4.5-5.5 parts ethyl-(2-hydroxyethyl)-dimethylazanium chloride, 4.5-5.5 parts dichlorozinc and 0.9-1.1 part ibrutinib; 0.9-1.1 part choline chloride, 0.9-1.1 part undec-10-enoic acid, 1.8-2.2 parts choline fluoride and 2.7-3.3 parts ibrutinib; 0.9-1.1 part choline chloride, 1.8-2.2 parts 1,3,4,5,6-pentahydroxyhexan-2-one, 0.9-1.1 part butane-1,4-diol and 4.5-5.5 parts ibrutinib; 0.9-1.1 part choline chloride, 2.7-3.3 parts octanoic acid, 1.8-2.2 parts ibrutinib, and 0.9-1.1 part benzyl(trimethyl)azanium chloride; 0.9-1.1 part camphor, 2.7-3.3 parts laurinol, 1.8-2.2 parts boric acid and 4.5-5.5 parts ibrutinib; 0.9-1.1 part choline chloride, 0.9-1.1 part nonanoic acid, 4.5-5.5 parts sodium acetate and 4.5-5.5 parts ibrutinib; 0.9-1.1 part borneol, 4.5-5.5 parts ethyl-(2-hydroxyethyl)-dimethylazanium chloride, 4.5-5.5 parts palmitic acid, 0.9-1.1 part lactose and 3.6-4.4 parts ibrutinib; 0.9-1.1 part choline chloride, 1.8-2.2 parts hexanoic acid, 2.7-3.3 parts ibrutinib, 2.7-3.3 parts boric acid and 0.9-1.1 part ethyl(2-hydroxyethyl)dimethylammonium chloride; and 0.9-1.1 part choline chloride, 2.7-3.3 parts ibrutinib, 1.8-2.2 parts 2-(diethylamino)ethanol hydrochloride and 2.7-3.3 parts hexanoic acid.

15. The eutectic mixture of claim 14 selected from the group consisting of: 1 part choline chloride, 5 parts laurinol 3 parts ibrutinib and 5 parts urea; 2 parts choline chloride, 5 parts ethyl-(2-hydroxyethyl)-dimethylazanium chloride, 5 parts dichlorozinc and 1 part ibrutinib; 1 part choline chloride, 1 part undec-10-enoic acid, 2 parts choline fluoride and 3 parts ibrutinib; 1 part choline chloride, 2 parts 1,3,4,5,6-pentahydroxyhexan-2-one, 1 part butane-1,4-diol and 5 parts ibrutinib; 1 part choline chloride, 3 parts octanoic acid, 2 parts ibrutinib, and 1 part benzyl(trimethyl)azanium chloride; 1 part camphor, 3 parts laurinol, 2 parts boric acid and 5 parts ibrutinib; 1 part choline chloride, 1 part nonanoic acid, 5 parts sodium acetate and 5 parts ibrutinib; 1 part borneol, 5 parts ethyl-(2-hydroxyethyl)-dimethylazanium chloride, 5 parts palmitic acid, 1 part lactose and 4 parts ibrutinib; 1 part choline chloride, 2 parts hexanoic acid, 3 parts ibrutinib, 3 parts boric acid and 1 part ethyl(2-hydroxyethyl)dimethylammonium chloride; and 1 part choline chloride, 3 parts ibrutinib, 2 parts 2-(diethylamino)ethanol hydrochloride and 3 parts hexanoic acid.

16. The eutectic mixture of claim 13 wherein said sildenafil derivative is defined by General Formula B: 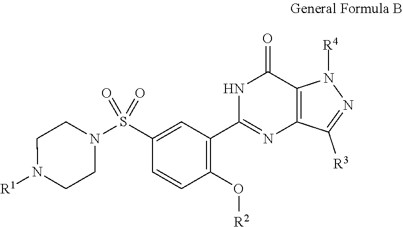
wherein:
R¹-R⁴ are independently selected from H, an alkyl of 1-10 carbons or an alkenyl of up to 10 carbons.

17. The eutectic mixture of claim 16 wherein R¹ is H or an alkyl of 1-3 carbons.

18. The eutectic mixture of claim 16 wherein R¹ is —CH₃.

19. The eutectic mixture of claim 16 wherein R² is H or an alkyl of 1-3 carbons.

20. The eutectic mixture of claim 19 wherein R¹ is —CH₂CH₃.

21. The eutectic mixture of claim 16 wherein R³ is H or an alkyl of 1-4 carbons.

22. The eutectic mixture of claim 21 wherein R³ is —CH₂CH₂CH₃.

23. The eutectic mixture of claim 16 wherein R⁴ is H or an alkyl of 1-3 carbons.

24. The eutectic mixture of claim 23 wherein R⁴ is —CH₃.

25. The eutectic mixture of claim 13 selected from the group consisting of: 2.7-3.3 parts choline chloride, 1.8-2.2 parts alpha-maltose 2.7-3.3 parts sildenafil and 2.7-3.3 parts (diethylamino)ethanol hydrochloride; 4.5-5.5 parts choline chloride, 3.6-4.4 parts laurinol, 2.7-3.3 parts benzyltriethylammonium chloride, 1.8-2.2 parts metilotic acid and 3.6-4.4 parts sildenafil; 4.5-5.5 parts choline fluoride, 1.8-2.2 parts glucose, 0.9-1.1 part laurinol, 1.8-2.2 parts tetradecanoic acid and 4.5-5.5 parts sildenafil; 0.9-1.1 part triethanolamine, 0.9-1.1 part ammonium rhodanide, 0.9-1.1 part sildenafil and 0.9-1.1 part 1,3-propanediol; 1.8-2.2 parts choline chloride, 1.8-2.2 parts galactose, 1 part DL-threonine, 1.8-2.2 parts menthol and 0.9-1.1 part sildenafil; 0.9-1.1 part ethylazanium chloride, 1.8-2.2 parts vanillin, 0.9-1.1 part alpha-D-glucopyranoside, 4.5-5.5 parts alpha-maltose and 0.9-1.1 part sildenafil; 3.6-4.4 parts choline chloride, 3.6-4.4 parts laurinol and 2.7-3.3 parts sildenafil; 0.9-1.1 part borneol, 3.6-4.4 parts D-ribose, 0.9-1.1 part laurinol and 4.5-5.5 parts sildenafil; 0.9-1.1 part choline chloride, 0.9-1.1 part glutamic acid and 4.5-5.5 parts sildenafil; and 0.9-1.1 part choline chloride, 1.8-2.2 parts sildenafil, 2.7-3.3 parts borneol, 2.7-3.3 parts hexafluoroisopropanol and 2.7-3.3 parts trimesic acid.

26. The eutectic mixture of claim 25 selected from the group consisting of: 3 parts choline chloride, 2 parts alpha-maltose 3 parts sildenafil and 3 parts (diethylamino)ethanol hydrochloride; 5 parts choline chloride, 4 parts laurinol, 3 parts benzyltriethylammonium chloride, 2 parts metilotic acid and 4 parts sildenafil; 5 parts choline fluoride, 2 parts glucose, 1 part laurinol, 2 parts tetradecanoic acid and 5 parts sildenafil; 1 part triethanolamine, 1 part ammonium rhodanide, 1 part sildenafil and 1 part 1,3, propanediol; 2 parts choline chloride, 2 parts galactose, 1 part DL-threonine, 2 parts menthol and 1 part sildenafil; 1 part ethylazanium chloride, 2 parts vanillin, 1 part alpha-D-glucopyranoside, 5 parts alpha-maltose and 1 part sildenafil; 4 parts choline chloride, 4 parts laurinol and 3 parts sildenafil; 1 part borneol, 4 parts D-ribose, 1 part laurinol and 5 parts sildenafil; 1 part choline chloride, 1 part glutamic acid and 5 parts sildenafil; and 1 part choline chloride, 2 parts sildenafil, 3 parts borneol 3 parts hexafluoroisopropanol and 3 parts trimesic acid.

27. The eutectic mixture of claim 13 wherein said ubiquinone derivative is defined by General Formula C: 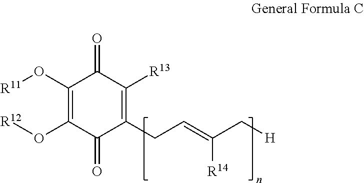
wherein R¹¹, R¹², R¹³ and R¹⁴ are independently selected from H, and an alkyl of 1-10 carbons; and
n is an integer of 1-15.

28. The eutectic mixture of claim 27 wherein R¹¹, R¹², R¹³ and R¹⁴ are independently an alkyl of 1-5 carbons.

29. The eutectic mixture of claim 20 wherein R¹¹, R¹², R¹³ and R¹⁴ are each —CH₃.

30. The eutectic mixture of claim 27 wherein n is 5-15.

31. The eutectic mixture of claim 30 wherein n is 9-11.

32. The eutectic mixture of claim 31 wherein n is 10.

33. The eutectic mixture of claim 13 selected from the group consisting of: 0.9-1.1 part choline chloride, 1.8-2.2 parts ubiquinone and 3.6-4.4 parts glycerol; 0.9-1.1 part ethylazanium chloride, 0.9-1.1 part butane-1,4-diol, 1.8-2.2 parts 1,1-dimethylurea, 0.9-1.1 part laurinol and 3.6-4.4 parts ubiquinone; 0.9-1.1 part choline chloride, 2.7-3.3 parts ubiquinone and 0.9-1.1 part glucose; 0.9-1.1 part choline chloride, 2.7-3.3 parts ubiquinone and 1.8-2.2 parts 1,3-dimethylurea; 0.9-1.1 part choline chloride, 1.8-2.2 parts maltose, 1.8-2.2 parts 1,3-dimethylurea, 0.9-1.1 part ubiquinone and 3.6-4.4 parts camphor; 4.5-5.5 parts camphor, 0.9-1.1 parts ubiquinone, 0.9-1.1 part butane-1,2,3,4-tetrol and 0.9-1.1 part ethane-1,2-diol; 4.5-5.5 parts choline chloride, 0.9-1.1 part ubiquinone and 1.8-2.2 parts dodecanoic acid; 0.9-1.1 part choline chloride, 3.6-4.4 parts alpha-maltose, 2.7-3.3 parts ubiquinone, and 3.6-4.4 parts phthalic acid; 1.8-2.2 parts choline chloride, 0.9-1.1 part 2-(trimethylazaniumyl)acetate, 5 parts ethyl-(2-hydroxyethyl)-dimethylazanium chloride, 4.5-5.5 parts ubiquinone and 2.7-3.3 parts nonanal; and 0.9-1.1 part camphor, 2.7-3.3 parts ubiquinone, 0.9-1.1 part ethylene glycol.

34. The eutectic mixture of claim 33 selected from the group consisting of: 1 part choline chloride, 2 parts ubiquinone and 4 parts glycerol; 1 part ethylazanium chloride, 1 part butane-1,4-diol, 2 parts 1,1-dimethylurea, 1 part laurinol and 4 parts ubiquinone; 1 part choline chloride, 3 parts ubiquinone and 1 part glucose; 1 part choline chloride, 3 parts ubiquinone and 2 parts 1,3-dimethylurea; 1 part choline chloride, 2 parts maltose, 2 parts 1,3-dimethylurea, 1 part ubiquinone and 4 parts camphor; 5 parts camphor, 1 parts ubiquinone, 1 part butane-1,2,3,4-tetrol and 1 part ethane-1,2-diol; 5 parts choline chloride, 1 part ubiquinone and 2 parts dodecanoic acid; 1 part choline chloride, 4 parts alpha-maltose, 3 parts ubiquinone, and 4 parts phthalic acid; 2 parts choline chloride, 1 part 2-(trimethylazaniumyl)acetate, 5 parts ethyl-(2-hydroxyethyl)-dimethylazanium chloride, 5 parts ubiquinone and 3 parts nonanal; and 1 part camphor, 3 parts ubiquinone, 1 part ethylene glycol.

35. The eutectic mixture of claim 13 wherein said tamoxifen derivative is defined by General Formula D: 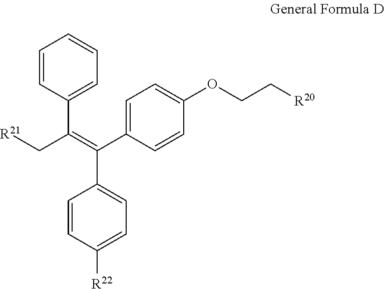
wherein:
R²⁰ is NR²³R²⁴ or OR²⁵;
R²¹ is CH₂R²⁶;
R²² is —H or —OH;
R²³ and R²⁴ are independently selected from H and an alkyl of 1-5 carbons and most preferably —CH₃;
R²⁵ is —H or an alkyl of 1-5 carbons and preferably H; and
R²⁶ is —H, —Cl or —OH.

36. The eutectic mixture of claim 35 wherein said tamoxifen derivative is selected from the group consisting of tamoxifen, endoxifen, N-desmethylamoxifen, didesmethylamoxifen, ospemifene and afimoxifene.

37. The eutectic mixture of claim 13 selected from the group consisting of: 0.9-1.1 part choline chloride, 4.5-5.5 parts tamoxifen, 2.7-3.3 parts hexitol, 0.9-1.1 part ethylene glycol and 0.9-1.1 part tetraethylazanium chloride; 0.9-1.1 part choline chloride, 0.9-1.1 part tamoxifen, 2.7-3.3 parts sodium acetate, 2.7-3.3 parts formic acid and 0.9-1.1 part butane-1,3-diol; 0.9-1.1 part choline chloride, 0.9-1.1 part tamoxifen, 1.8-2.2 parts choline fluoride, 4.5-5.5 parts hexanoic acid and 1.8-2.2 parts 2-aminoethanol; 0.9-1.1 part choline chloride, 3.6-4.4 parts diethyl(2-hydroxyethyl) azanium chloride, 2.7-3.3 parts diethanolamine and 1.8-2.2 parts tamoxifen; 0.9-1.1 part ethylazanium chloride, 0.9-1.1 part 1,2-butanediol, 1.8-2.2 parts methanol, 2.7-3.3 parts laurinol and 4.5-5.5 parts tamoxifen; 1.8-2.2 parts choline chloride, 1.8-2.2 parts tamoxifen and 2.7-3.3 parts choline fluoride; 2.7-3.3 parts choline chloride, 0.9-1.1 part tamoxifen and 2.7-3.3 parts glycerol; 2.7-3.3 parts choline chloride, 3.6-4.4 parts tamoxifen and 2.7-3.3 parts carnitine; 2.7-3.3 parts choline chloride, 0.9-1.1 part tamoxifen and 3.6-4.4 parts butane-1,3-diol; and 0.9-1.1 part choline chloride, 0.9-1.1 part hexanoic acid and 2.7-3.3 parts tamoxifen.20. The eutectic mixture of claim 19 selected from the group consisting of: 1 part choline chloride, 5 parts tamoxifen, 3 parts hexitol, 1 part ethylene glycol and 1 part tetraethylazanium chloride; 1 part choline chloride, 1 part tamoxifen, 3 parts sodium acetate, 3 parts formic acid and 1 part butane-1,3-diol; 1 part choline chloride, 1 part tamoxifen, 2 parts choline fluoride, 5 parts hexanoic acid and 2 parts 2-aminoethanol; 1 part choline chloride, 4 parts diethyl(2-hydroxyethyl) azanium chloride, 3 parts diethanolamine and 2 parts tamoxifen; 1 part ethylazanium chloride, 1 part 1,2-butanediol, 2 parts methanol, 3 parts laurinol and 5 parts tamoxifen; 2 parts choline chloride, 2 parts tamoxifen and 3 parts choline fluoride; 3 parts choline chloride, 1 part tamoxifen and 3 parts glycerol; 3 parts choline chloride, 4 parts tamoxifen and 3 parts carnitine; 3 parts choline chloride, 1 part tamoxifen and 4 parts butane-1,3-diol; and 1 part choline chloride, 1 part hexanoic acid and 3 parts tamoxifen.

38. The eutectic mixture of claim 1 further comprising camphor or nonanal.

39. A pharmaceutical composition comprising:
a eutectic mixture comprising:
a pharmaceutically active material and a least one compound selected from an amine or alkyl amine and an alcohol or a carboxylic acid.
wherein said eutectic mixture has a melting point low enough to remain liquid at ambient temperature.

40. The pharmaceutical composition of claim 39 wherein said eutectic mixture comprises an amine or alkyl amine.

41. The pharmaceutical composition of claim 40 wherein said amine is urea.

42. The pharmaceutical composition of claim 40 wherein said alkyl amine is an alkynol amine.

43. The pharmaceutical composition of claim 42 wherein said alkyl amine is selected from the group consisting of 1-(diethylamino)ethanol hydrochloride and threonine.

44. The pharmaceutical composition of claim 40 wherein said alkyl amine is selected from the group consisting of azanium halides; 2-[bis(2-hydroxyethyl)amino]ethanol, 2-(2-hydroxyethylamino)ethanol, tetraethylamine chloride, 1,3 dimethyl urea, trimethyl glycine (betaine), ethyl(2-hydroxyethyl)dimethylammonium chloride and ethanol amine.

45. The pharmaceutical composition of claim 44 wherein said azanium halides is selected from the group consisting of ethylazanium chloride, choline halide and particularly choline chloride, ethyl-(2-hydroxyethyl)-dimethylazanium chloride, benzyl(triethyl)azanium chloride, benzyl(trimethyl)azanium chloride and tetrabutylazanium chloride; 2-[bis(2-hydroxyethyl)amino]ethanol, 2-(2-hydroxyethylamino)ethanol, tetraethylamine chloride, 1,3 dimethyl urea, trimethyl glycine (betaine) and ethanol amine.

46. The pharmaceutical composition of claim 39 wherein said eutectic mixture comprises an alcohol.

47. The pharmaceutical composition of claim 46 wherein said alcohol is selected from a mono-alcohol and a polyol.

48. The pharmaceutical composition of claim 47 wherein said alcohol is selected from the group consisting of propane-1-ol, hexafluoroisopropanolmenthol, tetradecane-1-ol, 1-tetradecanol, borneol, vanillin, ethylene glycol, glycerol, butane-1,2-diol, 1,4 butanediol, 1,3-propanediol, 1,6-hexanediol, 1,3,4,5,6-pentahydroxyhexan-2-one, pentinol, ribose, glucose, galactose, lactose, maltose and alpha-D-glucopyranoside.

49. The pharmaceutical composition of claim 39 wherein said eutectic mixture comprises a carboxylic acid.

50. The pharmaceutical composition of claim 49 wherein said carboxylic acid is selected from the group consisting of tetradecanoic acid, 2-aminopentanedioic acid, lauric acid, nonanoic acid, formic acid, dodecanoic acid, adipic acid, stearic acid, undec-10-enoic acid, glutamic acid, palmitic acid, hexanoic acid, trimesic acid, octanoic acid, sorbose and laurinol.

51. The pharmaceutical composition of claim 39 wherein said pharmaceutically active material is selected from the group consisting of ibrutinib, a sildenafil derivative, a ubiquinone derivative and a tamoxifen derivative.

52. The pharmaceutical composition of claim 51 wherein said eutectic mixture is selected from the group consisting of: 0.9-1.1 part choline chloride, 4.5-5.5 parts laurinol, 2.7-3.3 parts ibrutinib and 4.5-5.5 parts urea; 1.8-2.2 parts choline chloride, 4.5-5.5 parts ethyl-(2-hydroxyethyl)-dimethylazanium chloride, 4.5-5.5 parts dichlorozinc and 0.9-1.1 part ibrutinib; 0.9-1.1 part choline chloride, 0.9-1.1 part undec-10-enoic acid, 1.8-2.2 parts choline fluoride and 2.7-3.3 parts ibrutinib; 0.9-1.1 part choline chloride, 1.8-2.2 parts 1,3,4,5,6-pentahydroxyhexan-2-one, 0.9-1.1 part butane-1,4-diol and 4.5-5.5 parts ibrutinib; 0.9-1.1 part choline chloride, 2.7-3.3 parts octanoic acid, 1.8-2.2 parts ibrutinib, and 0.9-1.1 part benzyl(trimethyl)azanium chloride; 0.9-1.1 part camphor, 2.7-3.3 parts laurinol, 1.8-2.2 parts boric acid and 4.5-5.5 parts ibrutinib; 0.9-1.1 part choline chloride, 0.9-1.1 part nonanoic acid, 4.5-5.5 parts sodium acetate and 4.5-5.5 parts ibrutinib; 0.9-1.1 part borneol, 4.5-5.5 parts ethyl-(2-hydroxyethyl)-dimethylazanium chloride, 4.5-5.5 parts palmitic acid, 0.9-1.1 part lactose and 3.6-4.4 parts ibrutinib; 0.9-1.1 part choline chloride, 1.8-2.2 parts hexanoic acid, 2.7-3.3 parts ibrutinib, 2.7-3.3 parts boric acid and 0.9-1.1 part ethyl(2-hydroxyethyl)dimethylammonium chloride; and 0.9-1.1 part choline chloride, 2.7-3.3 parts ibrutinib, 1.8-2.2 parts 2-(diethylamino)ethanol hydrochloride and 2.7-3.3 parts hexanoic acid.

53. The pharmaceutical composition of claim 52 wherein said eutectic mixture is selected from the group consisting of: 1 part choline chloride, 5 parts laurinol 3 parts ibrutinib and 5 parts urea; 2 parts choline chloride, 5 parts ethyl-(2-hydroxyethyl)-dimethylazanium chloride, 5 parts dichlorozinc and 1 part ibrutinib; 1 part choline chloride, 1 part undec-10-enoic acid, 2 parts choline fluoride and 3 parts ibrutinib; 1 part choline chloride, 2 parts 1,3,4,5,6-pentahydroxyhexan-2-one, 1 part butane-1,4-diol and 5 parts ibrutinib; 1 part choline chloride, 3 parts octanoic acid, 2 parts ibrutinib, and 1 part benzyl(trimethyl)azanium chloride; 1 part camphor, 3 parts laurinol, 2 parts boric acid and 5 parts ibrutinib; 1 part choline chloride, 1 part nonanoic acid, 5 parts sodium acetate and 5 parts ibrutinib; 1 part borneol, 5 parts ethyl-(2-hydroxyethyl)-dimethylazanium chloride, 5 parts palmitic acid, 1 part lactose and 4 parts ibrutinib; 1 part choline chloride, 2 parts hexanoic acid, 3 parts ibrutinib, 3 parts boric acid and 1 part ethyl(2-hydroxyethyl)dimethylammonium chloride; and 1 part choline chloride, 3 parts ibrutinib, 2 parts 2-(diethylamino)ethanol hydrochloride and 3 parts hexanoic acid.

54. The pharmaceutical composition of claim 51 wherein said sildenafil derivative is defined by General Formula B: 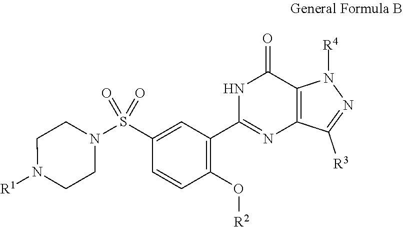
wherein:
R¹-R⁴ are independently selected from H, an alkyl of 1-10 carbons or an alkenyl of up to 10 carbons.

55. The pharmaceutical composition of claim 54 wherein R¹ is H or an alkyl of 1-3 carbons.

56. The pharmaceutical composition of claim 55 wherein R¹ is —CH₃.

57. The pharmaceutical composition of claim 54 wherein R² is H or an alkyl of 1-3 carbons.

58. The pharmaceutical composition of claim 55 wherein R¹ is —CH₂CH₃.

59. The pharmaceutical composition of claim 54 wherein R³ is H or an alkyl of 1-4 carbons.

60. The pharmaceutical composition of claim 59 wherein R³ is —CH₂CH₂CH₃.

61. The pharmaceutical composition of claim 54 wherein R⁴ is H or an alkyl of 1-3 carbons.

62. The pharmaceutical composition of claim 61 wherein R⁴ is —CH₃.

63. The pharmaceutical composition of claim 51 wherein said eutectic mixture is selected from the group consisting of: 2.7-3.3 parts choline chloride, 1.8-2.2 parts alpha-maltose 2.7-3.3 parts sildenafil and 2.7-3.3 parts (diethylamino)ethanol hydrochloride; 4.5-5.5 parts choline chloride, 3.6-4.4 parts laurinol, 2.7-3.3 parts benzyltriethylammonium chloride, 1.8-2.2 parts metilotic acid and 3.6-4.4 parts sildenafil; 4.5-5.5 parts choline fluoride, 1.8-2.2 parts glucose, 0.9-1.1 part laurinol, 1.8-2.2 parts tetradecanoic acid and 4.5-5.5 parts sildenafil; 0.9-1.1 part triethanolamine, 0.9-1.1 part ammonium rhodanide, 0.9-1.1 part sildenafil and 0.9-1.1 part 1,3-propanediol; 1.8-2.2 parts choline chloride, 1.8-2.2 parts galactose, 1 part DL-threonine, 1.8-2.2 parts menthol and 0.9-1.1 part sildenafil; 0.9-1.1 part ethylazanium chloride, 1.8-2.2 parts vanillin, 0.9-1.1 part alpha-D-glucopyranoside, 4.5-5.5 parts alpha-maltose and 0.9-1.1 part sildenafil; 3.6-4.4 parts choline chloride, 3.6-4.4 parts laurinol and 2.7-3.3 parts sildenafil; 0.9-1.1 part borneol, 3.6-4.4 parts D-ribose, 0.9-1.1 part laurinol and 4.5-5.5 parts sildenafil; 0.9-1.1 part choline chloride, 0.9-1.1 part glutamic acid and 4.5-5.5 parts sildenafil; and 0.9-1.1 part choline chloride, 1.8-2.2 parts sildenafil, 2.7-3.3 parts borneol, 2.7-3.3 parts hexafluoroisopropanol and 2.7-3.3 parts trimesic acid.

64. The pharmaceutical composition of claim 63 wherein said eutectic mixture is selected from the group consisting of: 3 parts choline chloride, 2 parts alpha-maltose 3 parts sildenafil and 3 parts (diethylamino)ethanol hydrochloride; 5 parts choline chloride, 4 parts laurinol, 3 parts benzyltriethylammonium chloride, 2 parts metilotic acid and 4 parts sildenafil; 5 parts choline fluoride, 2 parts glucose, 1 part laurinol, 2 parts tetradecanoic acid and 5 parts sildenafil; 1 part triethanolamine, 1 part ammonium rhodanide, 1 part sildenafil and 1 part 1,3, propanediol; 2 parts choline chloride, 2 parts galactose, 1 part DL-threonine, 2 parts menthol and 1 part sildenafil; 1 part ethylazanium chloride, 2 parts vanillin, 1 part alpha-D-glucopyranoside, 5 parts alpha-maltose and 1 part sildenafil; 4 parts choline chloride, 4 parts laurinol and 3 parts sildenafil; 1 part borneol, 4 parts D-ribose, 1 part laurinol and 5 parts sildenafil; 1 part choline chloride, 1 part glutamic acid and 5 parts sildenafil; and 1 part choline chloride, 2 parts sildenafil, 3 parts borneol 3 parts hexafluoroisopropanol and 3 parts trimesic acid.

65. The pharmaceutical composition of claim 51 wherein said ubiquinone derivative is defined by General Formula C: 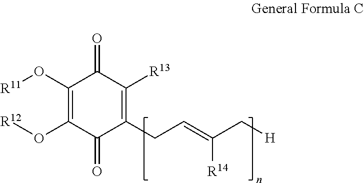
wherein R¹¹, R¹², R¹³ and R¹⁴ are independently selected from H, and an alkyl of 1-10 carbons; and n is an integer of 1-15.

66. The pharmaceutical composition of claim 65 wherein R¹¹, R¹², R¹³ and R¹⁴ are independently an alkyl of 1-5 carbons.

67. The pharmaceutical composition of claim 66 wherein R¹¹, R¹², R¹³ and R¹⁴ are each —CH₃.

68. The pharmaceutical composition of claim 65 wherein n is 5-15.

69. The pharmaceutical composition of claim 68 wherein n is 9-11.

70. The pharmaceutical composition of claim 69 wherein n is 10.

71. The pharmaceutical composition of claim 51 wherein said eutectic mixture is selected from the group consisting of: 0.9-1.1 part choline chloride, 1.8-2.2 parts ubiquinone and 3.6-4.4 parts glycerol; 0.9-1.1 part ethylazanium chloride, 0.9-1.1 part butane-1,4-diol, 1.8-2.2 parts 1,1-dimethylurea, 0.9-1.1 part laurinol and 3.6-4.4 parts ubiquinone; 0.9-1.1 part choline chloride, 2.7-3.3 parts ubiquinone and 0.9-1.1 part glucose; 0.9-1.1 part choline chloride, 2.7-3.3 parts ubiquinone and 1.8-2.2 parts 1,3-dimethylurea; 0.9-1.1 part choline chloride, 1.8-2.2 parts maltose, 1.8-2.2 parts 1,3-dimethylurea, 0.9-1.1 part ubiquinone and 3.6-4.4 parts camphor; 4.5-5.5 parts camphor, 0.9-1.1 parts ubiquinone, 0.9-1.1 part butane-1,2,3,4-tetrol and 0.9-1.1 part ethane-1,2-diol; 4.5-5.5 parts choline chloride, 0.9-1.1 part ubiquinone and 1.8-2.2 parts dodecanoic acid; 0.9-1.1 part choline chloride, 3.6-4.4 parts alpha-maltose, 2.7-3.3 parts ubiquinone, and 3.6-4.4 parts phthalic acid; 1.8-2.2 parts choline chloride, 0.9-1.1 part 2-(trimethylazaniumyl)acetate, 5 parts ethyl-(2-hydroxyethyl)-dimethylazanium chloride, 4.5-5.5 parts ubiquinone and 2.7-3.3 parts nonanal; and 0.9-1.1 part camphor, 2.7-3.3 parts ubiquinone, 0.9-1.1 part ethylene glycol.

72. The pharmaceutical composition of claim 71 wherein said eutectic mixture is selected from the group consisting of: 1 part choline chloride, 2 parts ubiquinone and 4 parts glycerol; 1 part ethylazanium chloride, 1 part butane-1,4-diol, 2 parts 1,1-dimethylurea, 1 part laurinol and 4 parts ubiquinone; 1 part choline chloride, 3 parts ubiquinone and 1 part glucose; 1 part choline chloride, 3 parts ubiquinone and 2 parts 1,3-dimethylurea; 1 part choline chloride, 2 parts maltose, 2 parts 1,3-dimethylurea, 1 part ubiquinone and 4 parts camphor; 5 parts camphor, 1 parts ubiquinone, 1 part butane-1,2,3,4-tetrol and 1 part ethane-1,2-diol; 5 parts choline chloride, 1 part ubiquinone and 2 parts dodecanoic acid; 1 part choline chloride, 4 parts alpha-maltose, 3 parts ubiquinone, and 4 parts phthalic acid; 2 parts choline chloride, 1 part 2-(trimethylazaniumyl)acetate, 5 parts ethyl-(2-hydroxyethyl)-dimethylazanium chloride, 5 parts ubiquinone and 3 parts nonanal; and 1 part camphor, 3 parts ubiquinone, 1 part ethylene glycol.

73. The pharmaceutical composition of claim 51 wherein said tamoxifen derivative is defined by General Formula D: 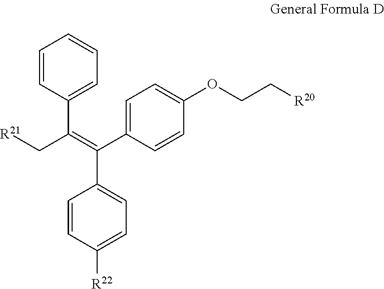
wherein:
R²⁰ is NR²³R²⁴ or OR²⁵;
R²¹ is CH₂R²⁶;
R²² is —H or —OH;
R²³ and R²⁴ are independently selected from H and an alkyl of 1-5 carbons and most preferably —CH₃;
R²⁵ is —H or an alkyl of 1-5 carbons and preferably H; and
R²⁶ is —H, —CI or —OH.

74. The pharmaceutical composition of claim 51 wherein said tamoxifen derivative is selected from the group consisting of tamoxifen, endoxifen, N-desmethylamoxifen, didesmethylamoxifen, ospemifene and afimoxifene.

75. The pharmaceutical composition of claim 74 wherein said eutectic mixture is selected from the group consisting of: 0.9-1.1 part choline chloride, 4.5-5.5 parts tamoxifen, 2.7-3.3 parts hexitol, 0.9-1.1 part ethylene glycol and 0.9-1.1 part tetraethylazanium chloride; 0.9-1.1 part choline chloride, 0.9-1.1 part tamoxifen, 2.7-3.3 parts sodium acetate, 2.7-3.3 parts formic acid and 0.9-1.1 part butane-1,3-diol; 0.9-1.1 part choline chloride, 0.9-1.1 part tamoxifen, 1.8-2.2 parts choline fluoride, 4.5-5.5 parts hexanoic acid and 1.8-2.2 parts 2-aminoethanol; 0.9-1.1 part choline chloride, 3.6-4.4 parts diethyl(2-hydroxyethyl) azanium chloride, 2.7-3.3 parts diethanolamine and 1.8-2.2 parts tamoxifen; 0.9-1.1 part ethylazanium chloride, 0.9-1.1 part 1,2-butanediol, 1.8-2.2 parts methanol, 2.7-3.3 parts laurinol and 4.5-5.5 parts tamoxifen; 1.8-2.2 parts choline chloride, 1.8-2.2 parts tamoxifen and 2.7-3.3 parts choline fluoride; 2.7-3.3 parts choline chloride, 0.9-1.1 part tamoxifen and 2.7-3.3 parts glycerol; 2.7-3.3 parts choline chloride, 3.6-4.4 parts tamoxifen and 2.7-3.3 parts carnitine; 2.7-3.3 parts choline chloride, 0.9-1.1 part tamoxifen and 3.6-4.4 parts butane-1,3-diol; and 0.9-1.1 part choline chloride, 0.9-1.1 part hexanoic acid and 2.7-3.3 parts tamoxifen.

76. The pharmaceutical composition of claim 75 wherein said eutectic mixture is selected from the group consisting of: 1 part choline chloride, 5 parts tamoxifen, 3 parts hexitol, 1 part ethylene glycol and 1 part tetraethylazanium chloride; 1 part choline chloride, 1 part tamoxifen, 3 parts sodium acetate, 3 parts formic acid and 1 part butane-1,3-diol; 1 part choline chloride, 1 part tamoxifen, 2 parts choline fluoride, 5 parts hexanoic acid and 2 parts 2-aminoethanol; 1 part choline chloride, 4 parts diethyl(2-hydroxyethyl) azanium chloride, 3 parts diethanolamine and 2 parts tamoxifen; 1 part ethylazanium chloride, 1 part 1,2-butanediol, 2 parts methanol, 3 parts laurinol and 5 parts tamoxifen; 2 parts choline chloride, 2 parts tamoxifen and 3 parts choline fluoride; 3 parts choline chloride, 1 part tamoxifen and 3 parts glycerol; 3 parts choline chloride, 4 parts tamoxifen and 3 parts carnitine; 3 parts choline chloride, 1 part tamoxifen and 4 parts butane-1,3-diol; and 1 part choline chloride, 1 part hexanoic acid and 3 parts tamoxifen.

77. The pharmaceutical composition of claim 39 further comprising menthol.

78. The pharmaceutical composition of claim 39 further comprising at least one of a carrier and a pharmaceutically active ingredient.

79. The pharmaceutical composition of claim 78 wherein said carrier is an oil.

80. The pharmaceutical composition of claim 78 wherein said pharmaceutically active ingredient is selected from the group consisting of a cough suppressant, an aromatic and an analgesic.

81. The pharmaceutical composition of claim 78 further comprising eucalyptol or eucalyptus oil.
